(19) United States
(12) Patent Application Publication
(10) Pub. No.: US 2007/0237848 A1
(43) Pub. Date: Oct. 11, 2007

(54) MORINDA CITRIFOLIA BASED COMPOSITIONS FOR TREATMENT OF ANTI-INFLAMMATORY DISEASES THROUGH INHIBITION OF COX-1, COX-2, INTERLEUKIN-1beta, INTERLEUKIN-6, TNF-alpha, HLE, AND iNOS

(76) Inventors: Brad Rawson, Orem, UT (US); Matthias-Heinrich Krenter, (US); Kim Asay, Alpine, UT (US); Afa K. Paln, American Fork, UT (US); Bing-Nan Zhou, Sandy, UT (US); Brett J. West, Orem, UT (US); Claude Jarake Jensen, Cedar Hills, UT (US)

Correspondence Address: KIRTON & McCONKIE;A PROFESSIONAL CORPORATION, ATTORNEYS AT LAW, 1800 EAGLE GATE TOWER, 60 EAST SOUTH TEMPLE STREET, SALT LAKE CITY, UT, 84111, US

(21) Appl. No.: 11/613,820

(22) Filed: Dec. 20, 2006

Related U.S. Application Data
(60) Provisional application No. 60/752,534, filed on Dec. 21, 2005.

(51) Int. Cl. A61K 36/74; A61P 29/00; A61P 35/00; C12N 9/99

(52) U.S. Cl. 424/776; 435/184

(57) ABSTRACT

Methods and compositions for inhibiting 5-Lipoxygenase, 15-Lipoxygenase, COX-1, COX-2, Interleukin-lβ, Interleukin-6, α, HLE, and iNOS. Methods and compositions for treating and preventing diseases, including inflammatory diseases and skin cancer. Compositions comprising processed Morinda citrifolia components, some of which include leaf extracts, leaf juice, and/or seed extracts.

### RELATED APPLICATIONS

This application claims priority to U.S. Provisional Patent Application Ser. No. 60/752,534, file Dec. 21, 2005.

### BACKGROUND OF THE INVENTION

1. Field of the Invention

The present invention relates to compositions comprising Morinda citrifolia, and methods for obtaining and using the same to inhibit 5-Lipoxygenase (5-LOX) and 15-Lipoxygenase (15-LOX),COX-1, COX-2, Interleukin-lβ, Interleukin-6, TNF-α, HLE, iNOS, inflammatory disease, and/or cancer.

2. Background and Related Art

Eicosanoids are continuously synthesized in membranes from 20-carbon fatty acid chains that contain at least three double bonds. There are four major classes of eicosanoids—prostaglandins, prostacyclins, thromboxanes, and leukotrienes—and they are all made mainly from arachidonic acid. The synthesis of all but the leukotrienes involves the enzyme cyclooxygenase (COX); the synthesis of leukotrienes involves the enzyme lipoxygenase (LOX). These synthetic pathways are targets for a large number of therapeutic drugs because eicosanoids play an important part in pain, fever, and inflammation. Corticosteroid hormones such as cortisone, for example, which inhibit the activity of the phospholipase in the first step of the eicosanoid synthesis pathway shown, are widely used clinically to treat noninfectious inflammatory diseases, such as some forms of arthritis. Nonsteroid anti-inflammatory drugs such as aspirin and ibuprofen, by contrast, block the first oxidation step, which is catalyzed by cyclooxygenase. Certain prostaglandins that are produced in large amounts in the uterus at the time of childbirth to stimulate the contraction of the uterine smooth muscle cells are widely used as pharmacological agents to induce abortion.

In addition to COX, the inhibition of cytokines, specifically Interleukin-lβ (IL-1β), Interleukin-6 (IL-6), and Tumor Necrosis Factor-α (TNF-α), has proven to have many clinical utilities. In general, cytokines are intercellular regulatory proteins that mediate a multiplicity of immunologic biological functions, and in certain pathological situations, particularly autoimmune diseases, chronic inflammatory diseases, and some leukemias, the production of cytokines are disregulated. The clinical benefits of IL-lβ, IL-6, and TNF-β will be discussed in turn.

IL-1 is a mediator of local and systemic inflammatory reactions, playing a pathogenetic role in septic shock and rheumatoid arthritis. Damage to the bone and cartilage caused by intense episodic synovitis in rheumatoid arthritis can be attributed IL-1, as well as other proinflammatory mediators such as TNF-α and IL-6, among other cytokines. Notably, IL-1 and TNF-α are particularly abundant in the cytokine profile of the synovial lining of the joint. Blockage of IL-1 has been shown to also be beneficial to other diseases, such as vasculitis, disseminated intravascular coagulation, osteoporosis, neurodegenerative disorders such as Alzheimer's disease, diabetes, lupus nephritis, immune complex glomerulonephritis and autoimmune diseases in general.

Enhanced IL-6 in serum has been found in a wide variety of trauma or inflammatory conditions, such as in serum of patients in trauma/surgery and in cerebral spinal fluid of patients with CNS infection or in vasculitis with CNS involvement. Additionally, IL-6 levels are enhanced in serum of patients with Crohn disease, with systemic lupus erythematosus, with alcoholic liver cirrhosis, and with Castleman disease. IL-6 is significantly enhanced in synovial fluid in rheumatoid arthritis. IL-6 has also been detected in multiple myeloma where it is expressed by tumor cells or stromal cells; in renal cell carcinoma, expressed by tumor cells; in cardiac myxoma patients, expressed by tumor cells and also found in serum.

The main physiological role of TNF is undoubtedly activation of the first-line reaction of the organism to microbal, parasitic, viral, or mechanical stress. It has an important role in antibacterial resistance and may be important in the host resistance against leishmaniasis. Plasmodial infection, as that of Malaria, results in an increase in circulating TNF levels, and anti-TNF antibodies have been found to protect against cerebral implications. Parasitic, bacterial, and some viral infections have become more pathogenic or fatal due to TNF in circulation. For example, CD4+ T cells latently infected by HIV can be stimulated to active viral replication by TNF. Additional studies have found that Graft-versus-host disease can be prevented or diminished by anti-TNF therapy or by treatments preventing the synthesis of endogenous TNF. In the case of rheumatoid arthritis, TNF is often present at the site of inflammation.

There are also many clinical benefits to inhibiting Human Leukocyte Elastase (HLE). HLE is a serine protease produced and released by PMNL, and because of its aggressive destructiveness, some investigators have found that HLE may play a role in several diseases, such as pulmonary emphysema, cystic fibrosis, chronic bronchitis, acute respiratory distress syndrome, glomerulonephritis and rheumatic arthritis. In emphysema, cystic fibrosis, and rheumatic arthritis it is believed that unbound HLE causes destruction of connective tissue, and therefore inhibition of HLE is desirable.

Inhibiting the inducible isoform of nitric oxide (iNOS) also exhibits clinical benefits. Nitric Oxide (NO ) plays a critical role during cerebral ischemia. NO is synthesized from L-arginine and oxygen by NO synthases (NOS). Small quanta of NO synthesized by constitutive NOS regulate a wide variety of physiological functions such as blood pressure, vascular tone, permeability, and neurotransmission. iNOS can be induced in microglia, astrocytes, endothelium, and vascular smooth muscle. Once expressed, it is continuously active, irrespective of intracellular calcium levels and leads to high output NO synthesis leading to cytotoxicity and inflammatory actions.

There are a plethora of molecular and biological mechanisms that contribute to inflammation-mediated cellular damage: The cerebral microcirculation becomes severely compromised by leukocyte plugging of small vessels. Neurons and macrophages may induce toxic enzymes such as iNOS or COX. iNOS is produced by invading neutrophils which may lead to increased NO production. With the use of pharmacological inhibition it has been unequivocally demonstrated that iNOS exerts neurotoxic effects during cerebral ischemia.

Over production of iNOS also contributes to septic shock, which is characterized by profound hypotension poorly responsive to fluid resuscitation and vasopressor therapy. In addition, NO also contributes to myocardial dysfunction and impaired cardiac output. In inflammation and infection, NO promotes the inflammatory response by enhancing cytokine release, such as TNF-α, and activation of COX with increased formation of prostaglandins.

The enzymes of the 5-LOX and 15-LOX pathway produce active metabolites from arachidonic acid that cause inflammation. This has been shown both by the identification of higher levels of leukotrienes in both acute and chronic inflammatory lesions coupled with the evidence of primary signs of inflammation when leukotrienes are added to tissue cultures. Leukotrienes are a family of lipid mediators involved in acute and chronic inflammation and allergic response diseases. They are the biologically active metabolites of arachidonic acid and have been implicated in the pathological manifestations of inflammatory diseases, including asthma, arthritis, psoriasis, and inflammatory bowel disease. The biosynthesis of leukotrienes (LT or LT's) begins with the oxygenation of arachidonic acid into an unstable epoxide known as LTA₄ (an intermediate central to the formation of leukotrienes) by the enzyme 5-lipoxygenase (5-LOX). LTA₄ can further be converted into the potent chemo attractant LTB₄ by the enzyme LTA₄ hydrolase or conjugated with glutathione (GSH) to produce LTC₄ by a specific microsomal GSH S-transferase (MGST) known as LTC₄ synthetase (LTC₄S). LTC₄ is the parent compound of the cysteinyl-leukotrienes (cys-LTs) that include LTC₄, LTD₄, and LTE₄. These three cysteinyl-leukotrienes are potent smooth muscle constricting agents, particularly in the respiratory and circulatory systems. These are mediated via at least two cell receptors, CysLT1 and CysLT2. The CysLT1 receptor is a G-protein-coupled receptor with seven transmembrane regions. There have been numerous amounts of data that has been collected, which clearly demonstrates that the CysLT's play a pivotal role in inflammatory and allergic response diseases, particularly asthma.

It has also been established that these lipid mediators have profound hemodynamic effects, constricting coronary blood vessels, resulting in a reduction of cardiac output efficiency. Moreover, CysLT's have been shown to induce the secretion of von Willebrand factor and surface expression of P-selectin in cultured HUVEC. Von Willebrand is a genetic disorder. The most common types, and those most familiar to people, are the hemophiliac diseases. These enzymes of the 5-LOX pathway produce active metabolites from arachidonic acid that cause inflammation. This has been shown both by the identification of higher levels of leukotrienes in both acute and chronic inflammatory lesions coupled with the evidence of primary signs of inflammation when leukotrienes are added to tissue cultures.

In addition, the cysteinyl LT's are predominantly secreted by eosinophils, mast cells, and macrophages, which cause vasodilatation, increase postcapillary venule permeability, and stimulate bronchoconstriction and mucous secretion. Furthermore, it has been observed that elevated leukotriene LTC₄ synthase activity was observed in peripheral blood granulocyte suspensions from patients with chronic myeloid leukemia (CML), and human bone marrow-derived myeloid progenitor cells. In asthma, the cysteinyl leukotrienes are present in alveolar lavage fluid of patients. Therefore, the presence of 5-LOX and leukotriene synthase are clinically important in the diagnosis of patients with bronchial asthma.

### SUMMARY OF THE INVENTION

Embodiments of the present invention relate to various methods of using specially processed components of the Indian Mulberry or Morinda citrifolia L. plant to inhibit the oxygenation and metabolizing of arachidonic acid into its leukotriene synthesized intermediates by inhibiting 5-Lipoxygenase (5-LOX), 15-Lipoxygenase (15-LOX) and the lipid mediators known as leukotrienes that contribute to the pathological manifestations of inflammatory diseases, namely, asthma, arthritis, psoriasis, and inflammatory bowel disease, as well as the treatment and prevention of these diseases.

Some embodiments of the invention include one or more processed Morinda citrifolia components such as: extract from the leaves of Morinda citrifolia, leaf hot water extract, processed Morinda citrifolia leaf ethanol extract, processed Morinda citrifolia leaf steam distillation extract, Morinda citrifolia fruit juice, Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate, freeze concentrated Morinda citrifolia fruit juice, and evaporated concentration of Morinda citrifolia fruit juice, whole Morinda citrifolia fruit in fresh, whole dried Morinda citrifolia fruit, powder or solvent extracted forms as well as enzyme treated Morinda citrifolia seeds, or any other processed Morinda citrifolia seed (i.e. roasting, blanching, microwaving, heat treatment, soaking in water or water solutions of various salts or chemical compounds), whole Morinda citrifolia fruit with blossoms or flowers attached, leaf extracts, leaf juice, and defatted and untreated seed extracts. Some of these methods include the steps of administering a Morinda citrifolia composition to a mammal to inhibit, prevent, or treat inflammatory diseases or cancer.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the above-recited and other advantages and features of the invention are understood, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIGS. 1A and 1B illustrate inhibition of 5-LOX activity by Morinda citrfolia seed extract, in particular FIG. 1A illustrates an embodiment of inhibition with untreated (sample labeled Vip_E_Moci'05_{—}87) seed extract and FIG. 1B illustrates inhibition with a defatted seed extract (sample labeled Vip_E_Moci'05_{—}88);

FIGS. 2A through 2F illustrate inhibition of 5-LOX by Morinda citrfolia seed extract with varying concentrations of ethanol used during the extraction process, wherein FIG. 2A illustrates inhibition by sample labeled Vip_E₁₃ Moci'05_{—}90, FIG. 2B illustrates inhibition by sample labeled Vip_E_Moci'05_{—}91; FIG. 2C illustrates inhibition by sample labeled Vip_E_Moci'05_{—}92; FIG. 2D illustrates inhibition by sample labeled Vip_E_Moci'05_{—}100; FIG. 2E illustrates inhibition by sample labeled Vip_E_Moci'05_{—}93; FIG. 2F illustrates inhibition by sample labeled Vip_E₁₃ Moci'05_{—}101;

FIGS. 3A and 3B illustrate inhibition of COX-1 by Morinda citrfolia seed extract, wherein FIG. 3A illustrates inhibition by sample labeled Vip_E_Moci'05_{—}100, and FIG. 3B illustrates inhibition by sample labeled Vip_E_Moci'05_{—}100.1;

FIGS. 4A and 4B illustrate inhibition of COX-1 by Morinda citrfolia extracts, wherein FIG. 4A illustrates inhibition by sample labeled Vip_E_Moci'05_{—}100, and FIG. 4B illustrates inhibition by sample labeled Vip_E_Moci'05_{—}100.1;

FIGS. 5A and 5B illustrate inhibition of TNF-α by Morinda citrfolia extracts, wherein FIG. 5A illustrates inhibition by sample labeled Vip_E₁₃ Moci'05_{—}100, and FIG. 5B illustrates inhibition by sample labeled Vip_E_Moci'05_{—}100.1;

FIGS. 6A and 6B illustrate inhibition of IL-6 by Morinda citrfolia seed extracts, wherein FIG. 6A illustrates inhibition with extract labeled Vip_E_Moci'05_{—}100, and FIG. 6B illustrates inhibition with sample labeled Vip_E_Moci'05_{—}100.1;

FIGS. 7A and 7B illustrate inhibition of HLE with Morinda citrfolia seed extracts, wherein FIG. 7A illustrates inhibition with sample labeled Vip_E_Moci'05_{—}100, and FIG. 7B illustrates inhibition with sample labeled Vip_E₁₃ Moci'05_{—}100.1;

FIGS. 8A and 8B illustrate inhibition of iNOS with Morinda citrfolia seed extracts, wherein FIG. 8A illustrates inhibition by sample labeled Vip_E_Moci'05_{—}100, and FIG. 8B illustrates inhibition with sample labeled Vip_E_Moci'05_{—}100.1;

FIG. 9 illustrates inhibition of iNOX by Morinda citrfolia seed extract; and

FIG. 10 illustrates the yield verses the Drug Solvent Ratio.

### DETAILED DESCRIPTION OF THE INVENTION

It will be readily understood that the components of the present invention, as generally described herein, could be arranged and designed in a wide variety of different configurations. Thus, the following more detailed description of embodiments of the compositions and methods of the present invention is not intended to limit the scope of the invention, as claimed, but is merely representative of the presently preferred embodiments of the invention. The scope of the invention is, therefore, indicated by the appended claims rather than by the foregoing description. All changes that come within the meaning and range of equivalency of the claims are to be embraced within their scope.

Embodiments of the present invention feature methods and compositions for inhibiting and for treating and preventing mammalian inflammatory diseases and skin cancer through the administration of a composition comprising components of the Indian Mulberry or Morinda citrifolia L. plant.

1. General Description of the Morinda citrifolia L. Plant

The Indian Mulberry or Morinda citrifolia plant, known scientifically as Morinda Citrifolia L. (“Morinda citrifolia”), is a shrub or small tree up to 10 m in height. The leaves are oppositely arranged with an elliptic to ovate form. The small white flowers are contained in a fleshy, globose, head-like cluster. The fruits are large, fleshy, and ovoid. At maturity, they are creamy-white and edible, but have an unpleasant taste and odor. The plant is native to Southeast Asia and has spread in early times to a vast area from India to eastern Polynesia. It grows randomly in the wild, and it has been cultivated in plantations and small individual growing plots. The Morinda citrifolia flowers are small, white, three to five lobed, tubular, fragrant, and about 1.25 cm long. The flowers develop into compound fruits composed of many small drupes fused into an ovoid, ellipsoid or roundish, lumpy body, with waxy, white, or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged 2-celled stones, each containing four seeds. When fully ripe, the fruit has a pronounced odor like rancid cheese. Although the fruit has been eaten by several nationalities as food, the most common use of the Morinda citrifolia plant has traditionally been as a red and yellow dye source.

2. Processing Morinda citrifolia Leaves

The leaves of the Morinda citrifolia plant are one possible component of the Morinda citrifolia plant that may be present in some compositions of the present invention. For example, some compositions comprise leaf extract and/or leaf juice as described further herein. Some compositions comprise a leaf serum that is comprised of both leaf extract and fruit juice obtained from the Morinda citrifolia plant. Some compositions of the present invention comprise leaf serum and/or various leaf extracts as incorporated into a nutraceutical product (“nutraceutical” herein referring to any drug or product designed to improve the health of living organisms such as human beings or mammals).

In some embodiments of the present invention, the Morinda citrifolia leaf extracts are obtained using the following process. First, relatively dry leaves from the Morinda citrifolia L. plant are collected, cut into small pieces, and placed into a crushing device—preferably a hydraulic press—where the leaf pieces are crushed. In some embodiments, the crushed leaf pieces are then percolated with an alcohol such as ethanol, methanol, ethyl acetate, or other alcohol-based derivatives using methods known in the art. Next, in some embodiments, the alcohol and all alcohol-soluble ingredients are extracted from the crushed leaf pieces, leaving a leaf extract that is then reduced with heat to remove all the liquid therefrom. The resulting dry leaf extract will herein be referred to as the “primary leaf extract.”

In some embodiments of the present invention, the primary leaf extract is pasteurized to at least partially sterilize the extract and destroy objectionable organisms. The primary leaf extract is pasteurized preferably at a temperature ranging from 70 to 80 degrees Celsius and for a period of time sufficient to destroy any objectionable organisms without major chemical alteration of the extract. Pasteurization may also be accomplished according to various radiation techniques or methods.

In some embodiments of the present invention, the pasteurized primary leaf extract is placed into a centrifuge decanter where it is centrifuged to remove or separate any remaining leaf juice therein from other materials, including chlorophyll. Once the centrifuge cycle is completed, the leaf extract is in a relatively purified state. This purified leaf extract is then pasteurized again in a similar manner as discussed above to obtain a purified primary leaf extract.

Preferably, the primary leaf extract, whether pasteurized and/or purified, is further fractionated into two individual fractions: a dry hexane fraction, and an aqueous methanol fraction. This is accomplished preferably via a gas chromatograph containing silicon dioxide and CH₂Cl₂-MeOH ingredients using methods well known in the art. In some embodiments of the present invention, the methanol fraction is further fractionated to obtain secondary methanol fractions. In some embodiments, the hexane fraction is further fractionated to obtain secondary hexane fractions.

One or more of the leaf extracts, including the primary leaf extract, the hexane fraction, methanol fraction, or any of the secondary hexane or methanol fractions may be combined with the fruit juice of the fruit of the Morinda citrifolia plant to obtain a leaf serum (the process of obtaining the fruit juice to be described further herein). In some embodiments, the leaf serum is packaged and frozen ready for shipment; in others, it is further incorporated into a nutraceutical product as explained herein.

3. Processing Morinda citrifolia Fruit

Some embodiments of the present invention include a composition comprising fruit juice of the Morinda citrifolia plant. Because the Morinda citrifolia fruit is for all practical purposes inedible, the fruit must be processed in order to make it palatable for human consumption and included in the compositions of the present invention. Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another product, frozen or pasteurized. In some embodiments of the present invention, the juice and pulp can be pureed into a homogenous blend to be mixed with other ingredients. Other processes include freeze drying the fruit and juice. The fruit and juice can be reconstituted during production of the final juice product. Still other processes may include air drying the fruit and juices prior to being masticated.

In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, but preferably for 2 to 3 days. The fruit is ripened or aged by being placed on equipment so that the fruit does not contact the ground. The fruit is preferably covered with a cloth or netting material during aging, but the fruit can be aged without being covered. When ready for further processing the fruit is light in color, such as a light green, light yellow, white or translucent color. The fruit is inspected for spoilage or for excessive green color and firmness. Spoiled and hard green fruit is separated from the acceptable fruit.

The ripened and aged fruit is preferably placed in plastic lined containers for further processing and transport. The containers of aged fruit can be held from 0 to 30 days, but preferably the fruit containers are held for 7 to 14 days before processing. The containers can optionally be stored under refrigerated conditions prior to further processing. The fruit is unpacked from the storage containers and is processed through a manual or mechanical separator. The seeds and peel are separated from the juice and pulp.

The juice and pulp can be packaged into containers for storage and transport. Alternatively, the juice and pulp can be immediately processed into a finished juice product. The containers can be stored in refrigerated, frozen, or room temperature conditions. The Morinda citrifolia juice and pulp are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.). Another product manufactured is Morinda citrifolia puree and puree juice, in either concentrate or diluted form. Puree is essentially the pulp separated from the seeds and is different than the fruit juice product described herein.

The product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment preferably consists of, but is not limited to, a centrifuge decanter, a screen filter with a size from 1 micron up to 2000 microns, more preferably less than 500 microns, a filter press, a reverse osmosis filtration device, and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The resulting pulp extract typically has a fiber content of 10 to 40 percent by weight. The resulting pulp extract is preferably pasteurized at a temperature of 181° F. (83° C.) minimum and then packed in drums for further processing or made into a high fiber product.

4. Processing Morinda citrifolia Seeds

Some Morinda citrifolia compositions of the present invention include seeds from the Morinda citrifolia plant. In some embodiments of the present invention, Morinda citrifolia seeds are processed by pulverizing them into a seed powder in a laboratory mill. In some embodiments, the seed powder is left untreated. In some embodiments, the seed powder is further defatted by soaking and stirring the powder in hexane—preferably for 1 hour at room temperature (Drug:Hexane—Ratio 1:10). The residue, in some embodiments, is then filtered under vacuum, defatted again (preferably for 30 minutes under the same conditions), and filtered under vacuum again. The powder may be kept overnight in a fume hood in order to remove the residual hexane.

Still further, in some embodiments of the present invention, the defatted and/or untreated powder is extracted, preferably with ethanol 50% (m/m) for 24 hours at room temperature at a drug solvent ratio of 1:2.

5. Processing Morinda citrifolia Oil

Some embodiments of the present invention may comprise oil extracted from the Morinda Citrifolia plant. The method for extracting and processing the oil is described in U.S. patent application Ser. No. 09/384,785, filed on Aug. 27, 1999 and issued as U.S. Pat. No. 6,214,351 on Apr. 10, 2001, which is incorporated by reference herein. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

6. Compositions and Their Use

The present invention features compositions and methods for inhibiting 5-LOX, 15-LOX, and/or skin cancer. The present invention also features compositions and methods for inhibiting the oxygenation of arachidonic acid into its leukotriene intermediate constituents for the purpose of treating and preventing inflammatory diseases. Embodiments of the present invention also comprise methods for internally introducing a Morinda citrifolia composition into the body of a mammal. Several embodiments of the Morinda citrifolia compositions comprise various different ingredients, each embodiment comprising one or more forms of a processed Morinda citrifolia component as taught and explained herein.

Compositions of the present invention may comprise any of a number of Morinda citrifolia components such as: leaf extract, leaf juice, leaf serum, fruit juice, fruit pulp, pulp extract, puree, seeds (whether defatted or untreated), and oil. Compositions of the present invention may also include various other ingredients. Examples of other ingredients include, but are not limited to: artificial flavoring, other natural juices or juice concentrates such as a natural grape juice concentrate or a natural blueberry juice concentrate; carrier ingredients; and others as will be further explained herein.

Any compositions having the leaf extract from the Morinda citrifolia leaves, may comprise one or more of the following: the primary leaf extract, the hexane fraction, methanol fraction, the secondary hexane and methanol fractions, the leaf serum, or the nutraceutical leaf product.

In some embodiments of the present invention, active ingredients or compounds of Morinda citrifolia components may be extracted out using various procedures and processes commonly known in the art. For instance, the active ingredients may be isolated and extracted out using alcohol or alcohol-based solutions, such as methanol, ethanol, and ethyl acetate, and other alcohol-based derivatives using methods known in the art. These active ingredients or compounds may be isolated and further fractioned or separated from one another into their constituent parts. Preferably, the compounds are separated or fractioned to identify and isolate any active ingredients that might help to prevent disease, enhance health, or perform other similar functions. In addition, the compounds may be fractioned or separated into their constituent parts to identify and isolate any critical or dependent interactions that might provide the same health-benefiting functions just mentioned.

Any components and compositions of Morinda citrifolia may be further incorporated into a nutraceutical product (again, “nutraceutical” herein referring to any drug or product designed to improve the health of living organisms such as human beings or mammals). Examples of nutraceutical products may include, but are not limited to: intravenous products, topical dermal products, wound healing products, skin care products, hair care products, beauty and cosmetic products (e.g., makeup, lotions, etc.), burn healing and treatment products, first-aid products, antibacterial products, lip balms and ointments, bone healing and treatment products, meat tenderizing products, anti-inflammatory products, eye drops, deodorants, antifungal products, arthritis treatment products, muscle relaxers, toothpaste, and various nutraceutical and other products as may be further discussed herein.

The compositions of the present invention may be formulated into any of a variety of embodiments, including oral compositions, topical dermal solutions, intravenous solutions, and other products or compositions.

Oral compositions may take the form of, for example, tablets, lozenges, aqueous or oily suspensions, dispersible powders or granules, emulsions, syrups, or elixirs. Compositions intended for oral use may be prepared according to any method known in the art, and such compositions may contain one or more agents such as sweetening agents, flavoring agents, coloring agents, and preserving agents. They may also contain one or more additional ingredients such as vitamins and minerals, etc. Tablets may be manufactured to contain one or more Morinda citrifolia components in admixture with non-toxic, pharmaceutically acceptable excipients that are suitable for the manufacture of tablets. These excipients may be, for example, inert diluents, granulating and disintegrating agents, binding agents, and lubricating agents. The tablets may be uncoated or they may be coated by known techniques to delay disintegration and absorption in the gastrointestinal tract and thereby provide sustained action over a longer period. For example, a time delay material such as glyceryl monostearate or glyceryl distearate may be used.

Aqueous suspensions may be manufactured to contain the Morinda citrifolia components in admixture with excipients suitable for the manufacture of aqueous suspensions. Examples of such excipients include, but are not limited to: suspending agents such as sodium carboxymethyl-cellulose, methylcellulose, hydroxy-propylmethycellulose, sodium alginate, polyvinyl-pyrrolidone, gum tragacanth and gum acacia; dispersing or wetting agents such as a naturally-occurring phosphatide like lecithin, or condensation products of an alkylene oxide with fatty acids such as polyoxyethylene stearate, or condensation products of ethylene oxide with long chain aliphatic alcohols such as heptadecaethylene-oxycetanol, or condensation products of ethylene oxide with partial esters derived from fatty acids and a hexitol such as polyoxyethylene sorbitor monooleate, or condensation products of ethylene oxide with partial esters derived from fatty acids and hexitol anhydrides such as polyethylene sorbitan monooleate.

Typical sweetening agents may include, but are not limited to: natural sugars derived from corn, sugar beets, sugar cane, potatoes, tapioca, or other starch-containing sources that can be chemically or enzymatically converted to crystalline chunks, powders, and/or syrups. Also, sweeteners can comprise artificial or high-intensity sweeteners, some of which may include aspartame, sucralose, stevia, saccharin, etc. The concentration of sweeteners may be between from 0 to 50 percent by weight of the Morinda citrifolia composition, and more preferably between about 1 and 5 percent by weight.

Typical flavoring agents can include, but are not limited to, artificial and/or natural flavoring ingredients that contribute to palatability. The concentration of flavors may range, for example, from 0 to 15 percent by weight of the Morinda citrifolia composition. Coloring agents may include food-grade artificial or natural coloring agents having a concentration ranging from 0 to 10 percent by weight of the Morinda citrifolia composition.

Typical nutritional ingredients may include vitamins, minerals, trace elements, herbs, botanical extracts, bioactive chemicals, and compounds at concentrations from 0 to 10 percent by weight of the Morinda citrifolia composition. Examples of vitamins include, but are not limited to, vitamins A, B1 through B12, C, D, E, Folic Acid, Pantothenic Acid, Biotin, etc. Examples of minerals and trace elements include, but are not limited to, calcium, chromium, copper, cobalt, boron, magnesium, iron, selenium, manganese, molybdenum, potassium, iodine, zinc, phosphorus, etc. Herbs and botanical extracts may include, but are not limited to, alfalfa grass, bee pollen, chlorella powder, Dong Quai powder, Ecchinacea root, Gingko Biloba extract, Horsetail herb, Indian mulberry, Shitake mushroom, spirulina seaweed, grape seed extract, etc. Typical bioactive chemicals may include, but are not limited to, caffeine, ephedrine, L-camitine, creatine, lycopene, etc.

The ingredients to be utilized in a topical dermal product may include any that are safe for internalizing into the body of a mammal and may exist in various forms, such as gels, lotions, creams, ointments, etc., each comprising one or more carrier agents. The ingredients or carrier agents incorporated into systemically (e.g., intravenously) administered compositions may also comprise any known in the art.

In one exemplary embodiment, a Morinda citrifolia composition of the present invention comprises one or more of a processed Morinda citrifolia component present in an amount by weight between about 0.01 and 100 percent by weight, and preferably between 0.01 and 95 percent by weight. Several embodiments of formulations are included in U.S. Pat. No. 6,214,351, issued on Apr. 10, 2001. However, these compositions are only intended to be exemplary, as one ordinarily skilled in the art will recognize other formulations or compositions comprising the processed Morinda citrifolia product.

In another exemplary embodiment, the internal composition comprises the ingredients of: processed Morinda citrifolia fruit juice or puree juice present in an amount by weight between about 0.1-80 percent; processed Morinda citrifolia oil present in an amount by weight between about 0.1-20 percent; and a carrier medium present in an amount by weight between about 20-90 percent. Morinda citrifolia puree juice or fruit juice may also be formulated with a processed Morinda citrifolia dietary fiber product present in similar concentrations.

### 7. EXAMPLES

The following examples illustrate some of the preventative and treatment effects of some Morinda citrifolia compositions of the present invention on 5-LOX, 15-LOX, COX-1, COX-2, Interleukinlβ, Interleukin-6, TNF-α, HLE,iNOS, inflammatory diseases, and/or cancer. These examples are not intended to be limiting in any way, but are merely illustrative of benefits, advantages, and remedial effects of some embodiments of the Morinda citrifolia compositions of the present invention.

### Example 1

A study was performed to measure the potential inhibitory effects of untreated and de-fatted Morinda citrifolia seed extracts on the activity of human 5-Lipoxygenase (5-LOX). Morinda citrifolia seeds were pulverized in a laboratory mill. Half of the resulting seed powder was left untreated, and the other half was defatted by soaking and stirring the powder in hexane for 1 hour at room temperature (Drug:Hexane—Ratio 1:10). After filtration under vacuum, the residue was defatted again for 30 minutes under the same conditions and filtered under vacuum again. In order to remove the residual hexane, the powder was kept overnight in a fume hood.

The defatted as well as the untreated powder was extracted with ethanol 50% (m/m) for 24 hours at room temperature at a drug solvent ratio of 1:2. The fluid extracts were directly used for the bioassays after filtration without further concentration steps. A stock solution of 15 mg/ml in ethanol 50% was prepared for the 5-LOX assay.

A Lipoxygenase Assay (1, 2) in human HL-60 cells was then performed as follows. Human HL-60 cells (myeloid leukemia, DSMZ No ACC 3) were kept at 37° C. in a humidified atmosphere with 5% CO₂ and cultured in complete RPMI 1640 medium supplemented with 10% fetal calf serum and 1% (v/v) penicillin/streptomycin solution. Cells were differentiated for 6 to 8 days with DMSO (1.2% v/v). The 5-LOX activity assay was carried out as described by C. F. Bennet, M. Y. Chiang, B. P. Monia, and S. T. Crooke in “Regulation of 5-lipoxygenase-activating protein expression in HL-60 cells,” Biochem. J. 289: 33-39. Briefly, differentiated cells were harvested, suspended in PBS containing Ca²⁺(1 mM) and glucose (1 mM) and distributed into a 96-well microtiter plate (1×10⁶ cells/well).

Stock solutions of test compounds in appropriate solvent were diluted with PBS. After pre-incubation with a sample or vehicle for 15 minutes at room temperature, the reaction was started by adding calcium ionophore A 23187 (5 μM) and arachidonic acid (10 μM). All values taken represented final values for the solvent concentrations. Negative controls were carried out without calcium ionophore stimulation. The assay mix (100 μl) was incubated for 15 minutes at 37° C. and terminated by adding 100 μl methanol containing HCl (1 M, 3% v/v) and placing the microtiter plate on ice. After centrifugation (340×g) for 10 minutes, the LTB₄ concentration in the supernatant was determined.

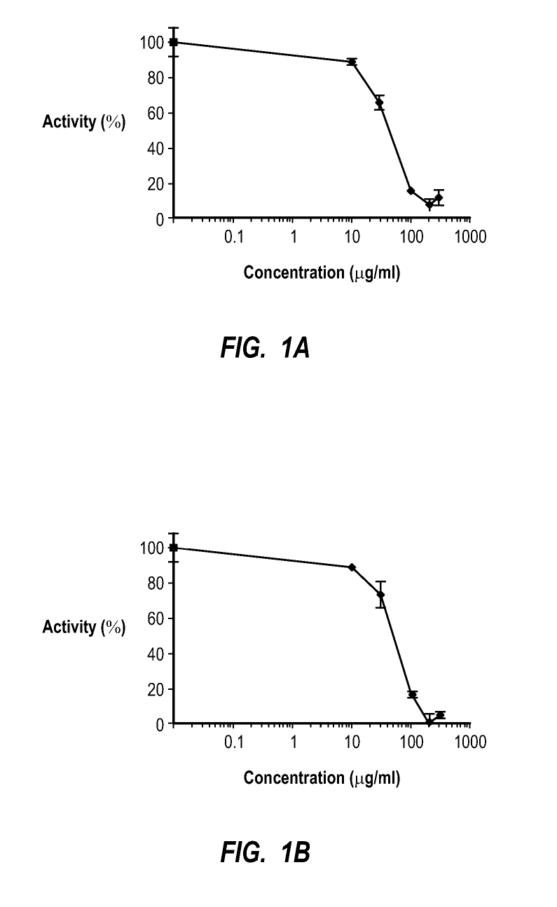

Effects of samples and reference compounds on the activity of 5-LOX were measured by determining the quantity of Leukotrien B₄ produced under assay conditions. The quantification of Leukotrien B₄ was performed with Enzyme Immuno Assay (EIA) Kit from Cayman No 520111 (LTB₄). The optical densities were measured at λ=415 mn. The quantities were calculated using a standard curve of at least 5 different concentrations. Sample points were measured as duplicates. Dose related inhibition values were expressed as a percentage of the positive control values. The following tables and charts and FIGS. 1A and 1B summarize the assay and the results.

Samples Used

| | Extraction | | |
|---|---|---|---|
| Plant part | solvent | Treatment | Sample number |
| Morinda citrifolia Seed | 50% | Untreated | Vip_E_Moci'05_87 |
| | ethanol | | |
| Morinda citrifolia Seed | 50% | De-fatted | Vip_E_Moci'05_88 |
| | ethanol | | |

IC₅₀ Values

| Sample number | IC₅₀ (μg/μl) |
|---|---|
| Vip_E_Moci'05_87 | 50 |
| Vip_E_Moci'05_88 | 60 |

| Standard reference agent | IC₅₀ (μM) |
|---|---|
| NDGA (nordihydroguaretic acid) | 0.1 |

Concentrations

| | Assay concentrations | Final solvent |
|---|---|---|
| Sample number | (μg/ml) | concentration |
| Vip_E_Moci'05_87 | 10, 30, 100, 200, 300 | 1% ethanol |
| Vip_E_Moci'05_88 | 10, 30, 100, 200, 300 | 1% ethanol |

Raw data of 5-LOX inhibition

| Vip_E_Moci'05_87 | | | Vip_E_Moci'05_88 | | |
|---|---|---|---|---|---|
| | t (15) | t (0) | | t (15) | t (0) |
| Concentration | LTB4 | LTB4 | Concentration | LTB4 | LTB4 |
| (μg/ml) | (pg/ml) | (pg/ml) | (μg/ml) | (pg/ml) | (pg/ml) |
| 300 | 3011 | 2499 | 300 | 3418 | 3026 |
| 300 | 3481 | 2793 | 300 | 3618 | 2964 |
| 200 | 2822 | * | 200 | 3517 | * |
| 200 | 3204 | * | 200 | 2800 | * |
| 100 | 3463 | 2379 | 100 | 4216 | 2911 |
| 100 | 3436 | 2447 | 100 | 4044 | 3082 |
| 30 | 6684 | * | 30 | 7787 | * |
| 30 | 6184 | * | 30 | 6859 | * |
| 10 | 7929 | * | 10 | 8137 | * |
| 10 | 7706 | * | 10 | 8032 | * |
| control | 8286 | 2230 | control | 8286 | 2230 |
| control | 7588 | 2156 | control | 7588 | 2156 |
| control | 8307 | * | control | 8307 | * |
| control | 8749 | * | control | 8749 | * |

In summary, both tested seed extracts of Morinda citrifolia clearly inhibited the activity of 5-LOX in vitro. No relevant difference was observed between the untreated and the de-fatted extracts. Vip_E_Moci'05_{—}87, the untreated extract, inhibited the 5-LOX activity with an IC₅₀ value of 50 μg/ml, and Vip_E_Moci'05₁₃ 88, the de-fatted extract, he 5-LOX activity with an IC₅₀ value of 60 μg/ml.

### Example 2

In another example, a pharmacological screening study of Morinda citrifolia in vitro was performed. The aim of this study was to measure the potential inhibitory effects of different ethanol extracts of Morinda citrifolia seeds on the activity of human 5-Lipoxygenase (5-LOX).

Morinda citrifolia seeds were pulverized in a laboratory mill. Half of the resulting see powder was left untreated, and the other half was defatted by soaking and stirring the powder in hexane for 1 hour at room temperature (Drug:Hexane—Ratio 1:10). After filtration under vacuum, the residue was defatted again for 30 minutes under the same conditions and filtered under vacuum again. In order to remove the residual hexane, the powder was kept overnight in a fume hood.

The defatted powder was extracted with different ethanol concentrations for 24 hours at room temperature at a drug solvent ratio of 1:2. The fluid extracts were directly used for the bioassays after filtration without further concentration steps.

A Lipoxygenase Assay (1, 2) in human HL-60 cells was then performed as follows. Human HL-60 cells (myeloid leukemia, DSMZ No ACC 3) were kept at 37° C. in a humidified atmosphere with 5% CO₂ and cultured in complete RPMI 1640 medium supplemented with 10% fetal calf serum and 1% (v/v) penicillin/streptomycin solution. Cells were differentiated for 6 to 8 days with DMSO (1.2% v/v). The 5-LOX activity assay was carried out as described by C. F. Bennet, M. Y. Chiang, B. P. Monia, and S. T. Crooke in “Regulation of 5-lipoxygenase-activating protein expression in HL-60 cells,” Biochem. J. 289: 33-39. Briefly, differentiated cells were harvested, suspended in PBS containing Ca²⁺(1 mM) and glucose (1 mM) and distributed into a 96-well microtiter plate (1×10₆ cells/well).

Stock solutions of test compounds in appropriate solvent were diluted with PBS. After pre-incubation with a sample or vehicle for 15 minutes at room temperature, the reaction was started by adding calcium ionophore A 23187 (5 μM) and arachidonic acid (10 μM). All values taken represented final values for the solvent concentrations. Negative controls were carried out without calcium ionophore stimulation. The assay mix (100 μl) was incubated for 15 minutes at 37° C. and terminated by adding 100 μl methanol containing HCl (1 M, 3% v/v) and placing the microtiter plate on ice. After centrifugation (340×g) for 10 minutes, the LTB₄ concentration in the supernatant was determined.

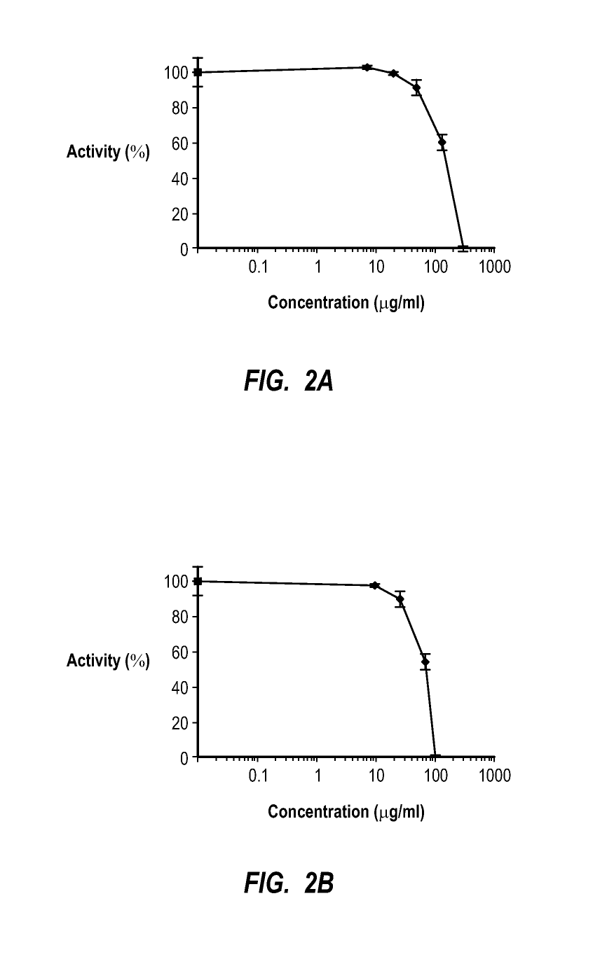

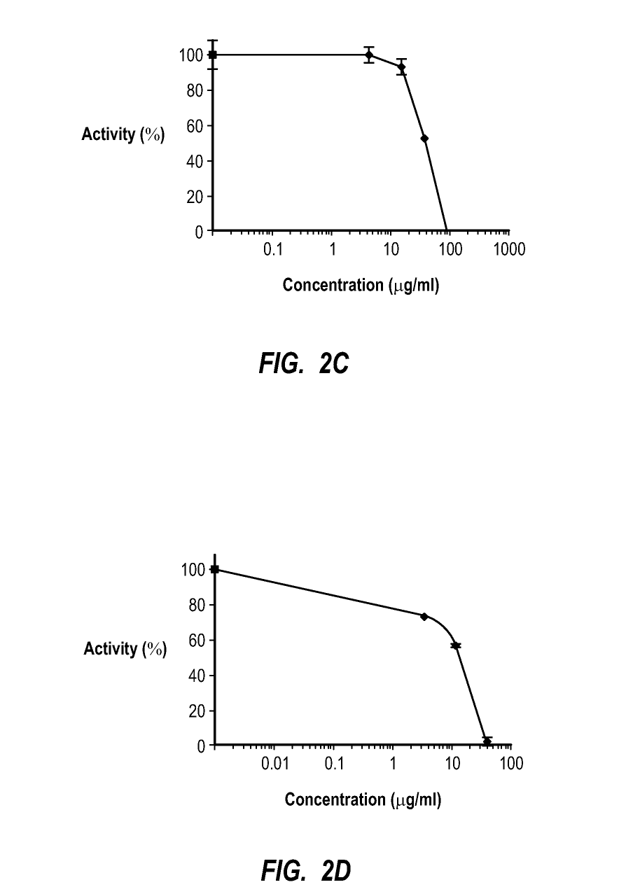

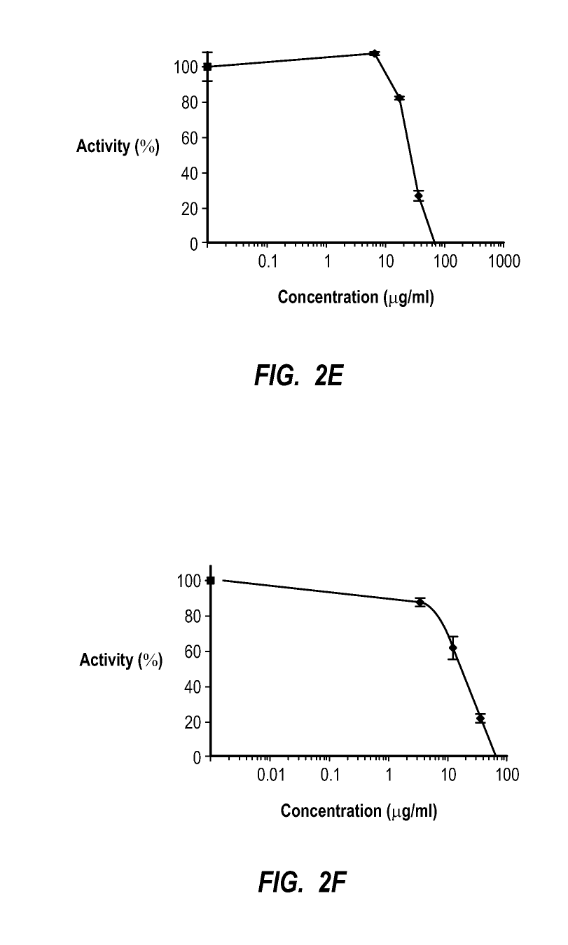

Effects of samples and reference compounds on the activity of 5-LOX were measured by determining the quantity of Leukotrien B₄ produced under assay conditions. The quantification of Leukotrien B₄ was performed with Enzyme Immuno Assay (EIA) Kit from Cayman No 520111 (LTB₄). The optical densities were measured at λ=415 nm. The quantities were calculated using a standard curve of at least 5 different concentrations. Sample points were measured as duplicates. Dose related inhibition values were expressed as a percentage of the positive control values. The following tables, charts and FIGS. 2A-2F summarize the assay and the results.

Samples Used

| | Extract type, | |
|---|---|---|
| Sample number* | Drug:Solventl:Ratio | Extract number |
| ViP_Moci'05_32 | ETOH 30% m/m, 1:2 | Vip_E_Moci'05_90 |
| ViP_Moci'05_32 | ETOH 50% m/m, 1:2 | Vip_E_Moci'05_91 |
| ViP_Moci'05_32 | ETOH 70% m/m, 1:2 | Vip_E_Moci'05_92 |
| ViP_Moci'05_32 | ETOH 90% m/m, 1:2 | Vip_E_Moci'05_93 |
| ViP_Moci'05_32 | ETOH 80% m/m, 1:2 | Vip_E_Moci'05_100 |
| ViP_Moci'05_32 | ETOH 96% m/m, 1:2 | Vip_E_Moci'05_101 |

*The samples comprised whole dried Morinda Citrifolia seeds.

IC₅₀ Values

| | Extraction | |
|---|---|---|
| Sample number | (% EtOH m/m) | IC₅₀ (μg/μl) |
| Vip_E_Moci'05_90 | 30 | 100 |
| Vip_E_Moci'05_91 | 50 | 40 |
| Vip_E_Moci'05_92 | 70 | 30 |
| Vip_E_Moci'05_100 | 80 | 10 |
| Vip_E_Moci'05_93 | 90 | 20 |
| Vip_E_Moci'05_101 | 96 | 14 |

| Standard reference agent | IC₅₀ (μM) |
|---|---|
| NDGA (nordihydroguaretic acid) | 0.5 |

Concentrations

| | Assay concentrations | Final solvent |
|---|---|---|
| Samples | (μg/ml) | concentration |
| All samples | 3, 10, 30 | 1% EtOH |

In summary, all seed extracts of Morinda citrifolia clearly inhibited the activity of 5-LOX in vitro. The degree of inhibition of the 5-LOX activity varied with ethanol content of the extraction solution. With increasing ethanol content of the extraction solution up to an EtOH content of 80%, the extracts displayed increasing inhibitory effects. At 80% EtOH content the extract ViP_E_Moci'05_{—}100 inhibited the 5-LOX activity with an IC50-value of 10 μg/ml. Lower or higher ethanol contents in the extraction solutions decreased the inhibitory effects of the extracts as indicated by higher IC₅₀ values.

A pharmacological screening study was performed to measure the activity spectrum of Morinda citrifolia seed extracts and to determine if prolonged storage has an influence on the biological activity of the extracts. To this end, the potential inhibitory effect of two extracts on the activity of Cyclooxygenase-1 (COX-1) and Cyclooxygenase-2 (COX-2) was measured. Specifically, the IC₅₀ values were measured on isolated enzymes for COX-1 and COX-2.

Morinda citrifolia seeds were pulverized in a laboratory mill. Half of the resulting seed powder was left untreated, and the other half was defatted by soaking and stirring the powder in hexane for 1 hour at room temperature (Drug:Hexane—Ratio 1:10). After filtration under vacuum, the residue was defatted again for 30 minutes under the same conditions and filtered under vacuum again. In order to remove the residual hexane, the powder was kept overnight in a fume hood.

The defatted as well as the untreated powder was extracted with ethanol 50% (m/m) for 24 hours at room temperature at a drug solvent ratio of 1:2. The fluid extracts were directly used for the bioassays after filtration without further concentration steps. A stock solution of 15 mg/ml in ethanol 50% was prepared for the 5-LOX assay.

Assays were then performed for COX-1 (ram seminal vesicles) and COX-2 (sheep placenta) as follows. After preincubation of the samples with the assay mixture for 15 minutes at room temperature, the reaction was started with arachidonic acid (10 μM). The incubation time was 3 minutes. The controls [t(0)] were performed with heat inactivated enzyme.

The effect of several concentrations of sample and reference compounds on the activity of COX was measured by determining the quantity of Prostaglandine E₂ (PGE₂) produced under the assay conditions.

The quantification of PGE₂ was performed with Enzyme Immuno Assay (EIA) Kits from Cayman No 514010. The optical densities were measured at λ=415 nm. The quantities were calculated using a standard curve of at least 5 different concentrations.

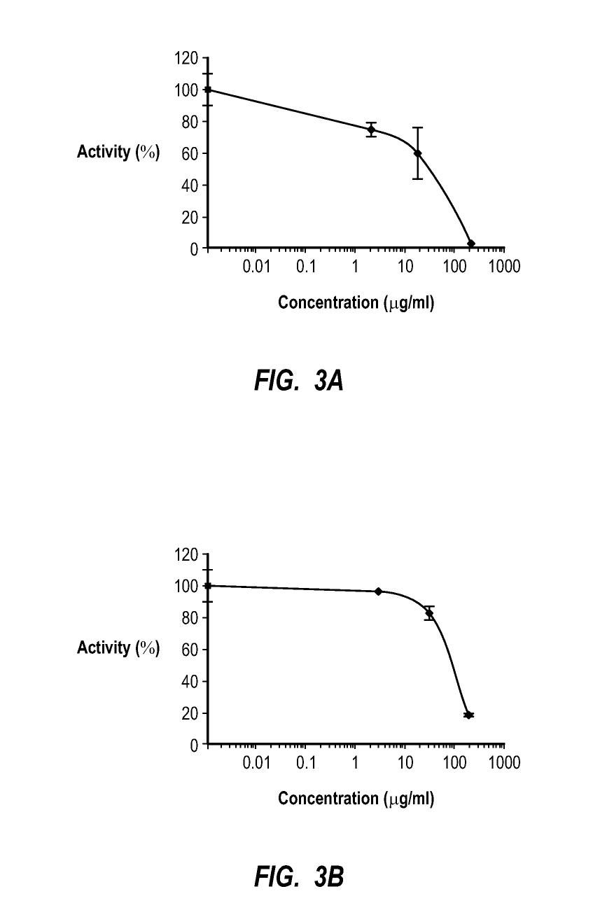

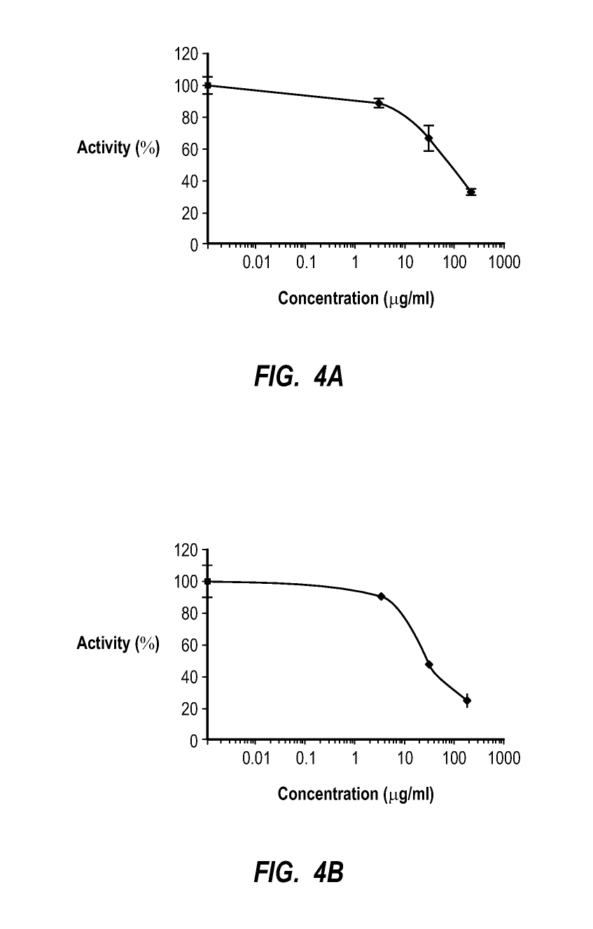

Each sample point was measured in duplicate. The dose related inhibition values were expressed as a percentage of the positive control values. The IC₅₀ values (corresponding to the sample concentration at which the inhibition level is 50%) were determined graphically. The following tables, charts, and FIGS. 3A, 3B, 4A and 4B summarize the assay and the results.

Samples Used
Concentrations

| | Assay | | |
|---|---|---|---|
| | concentrations | Serial | Final solvent |
| Sample number | (μg/ml) | dilution in | concentration |
| Vip_E_Moci'05_100 | 3, 30, 227 | Ethanol 10% | Ethanol 1% |
| Vip_E_Moci'05_100.1 | 3, 30, 181 | | |

COX-1
IC₅₀ Values of COX-1

| Sample number | IC₅₀ (μg/μl) |
|---|---|
| Vip_E_Moci'05_100 | 50 |
| Vip_E_Moci'05_100.1 | 80 |

| Standard reference agent | IC₅₀ (μM) |
|---|---|
| Indomethacin | 0.3 |

Raw Data of COX-1 Inhibition

| Vip_E_Moci'05_100 | | | Vip_E_Moci'05_100.1 | | |
|---|---|---|---|---|---|
| | t (15) | t (0) | | t (15) | t (0) |
| Concentration | PGE₂ | PGE₂ | Concentration | PGE₂ | PGE₂ |
| (μg/ml) | (pg/ml) | (pg/ml) | (μg/ml) | (pg/ml) | (pg/ml) |
| 227 | 667 | 663 | 181 | 706 | 663 |
| 227 | 339 | * | 181 | 699 | * |
| 30 | 926 | * | 30 | 954 | * |
| 30 | 794 | * | 30 | 928 | * |
| 3 | 906 | 631 | 3 | 983 | 574 |
| 3 | 931 | * | 3 | 981 | * |
| Control | 946 | 663 | Control | 946 | 663 |
| Control | 983 | 574 | Control | 983 | 574 |
| Control | 1011 | * | Control | 1011 | * |

COX-2
IC₅₀ Values of COX-2

| Sample number | IC₅₀ (μg/μl) |
|---|---|
| Vip_E_Moci'05_100 | 80 |
| Vip_E_Moci'05_100.1 | 25 |

| Standard reference agent | IC₅₀ (μM) |
|---|---|
| Indomethacin | 6 |

Raw Data of COX-2 Inhibition

| Vip_E_Moci'05_100 | | | Vip_E_Moci'05_100.1 | | |
|---|---|---|---|---|---|
| | t (15) | t (0) | | t (15) | t (0) |
| Concentration | PGE₂ | PGE₂ | Concentration | PGE₂ | PGE₂ |
| (μg/ml) | (pg/ml) | (pg/ml) | (μg/ml) | (pg/ml) | (pg/ml) |
| 227 | 668 | 579 | 181 | 716 | 643 |
| 227 | 652 | * | 181 | 691 | * |
| 30 | 826 | * | 30 | 795 | * |
| 30 | 763 | * | 30 | 781 | * |
| 3 | 894 | 477 | 3 | 943 | 565 |
| 3 | 879 | * | 3 | 940 | * |
| Control | 941 | 643 | Control | 941 | 643 |
| Control | 952 | 565 | Control | 952 | 565 |
| Control | 985 | * | Control | 985 | * |

In summary, the seed extracts of Morinda citrifolia clearly inhibited the activity of COX -1 in vitro. The degree of inhibition of the COX-1 activity varied with an IC₅₀ value from 50 μg/ml for the ViP_E_Moci'05_{—}100 extract and 80 μg/ml for the ViP_E_Moci'05_{—}100.1 extract. As for the activity of COX-2, ViP_E_Moci'05_{—}100.1 showed a stronger inhibition with an IC₅₀value of 25 μg/ml than ViP_E_Moci'05₁₃ 100 with an IC₅₀ value of only 80 μg/ml.

### Example 3

In another example, a pharmacological screening study was performed to measure the activity spectrum of Morinda citrifolia seed extracts and to determine if prolonged storage has an influence on the biological activity of the extracts. This study measured the potential inhibitory effect of two Morinda citrifolia extracts on the activity of cytokines Interleukin-1β, Interleukin-6, and TNF-α. Specifically, the IC₅₀ values were measured on human monocytes (differentiated THP-1 cells) for the cytokines.

Morinda citrifolia seeds were pulverized in a laboratory mill. Half of the resulting seed powder was left untreated, and the other half was defatted by soaking and stirring the powder in hexane for 1 hour at room temperature (Drug:Hexane—Ratio 1:10). After filtration under vacuum, the residue was defatted again for 30 minutes under the same conditions and filtered under vacuum again. In order to remove the residual hexane, the powder was kept overnight in a fume hood.

The defatted powder was extracted with different ethanol concentrations for 24 hours at room temperature at a drug solvent ratio of 1:2. The fluid extracts were directly used for the bioassays after filtration without further concentration steps.

A Cytokine Assay (α, IL-1βand IL-6) in human THP-1 cells was then performed as follows. The samples were preincubated for 30 minutes at 37° C. with cells (human THP-1) previously differentiated with PMA (5×104 cells/ml for α, 104 cells/ml for IL-1β, 5×105 cells/ml for Il -6). The reaction was started with LPS (1 μg/ml) and the incubation was performed over 24 hours at 37° C. Negative controls [t(0)] were carried out with the assay mixture without LPS stimulation.

The quantification of TNF-α, IL-1βand IL-6 was performed with Enzyme Immuno Assay (EIA) Kits from Cayman No 589201 (TNF-α), No: 583311 (IL-1β) and No: 583361 (IL-6). The optical densities were measured at λ=415 nm. The quantities were calculated using a standard curve of at least 5 different concentrations.

Each sample point was measured in duplicate. The dose related inhibition values were expressed as a percentage of the positive control values. The IC₅₀ values (corresponding to the sample concentration at which the inhibition level is 50%) were determined graphically.

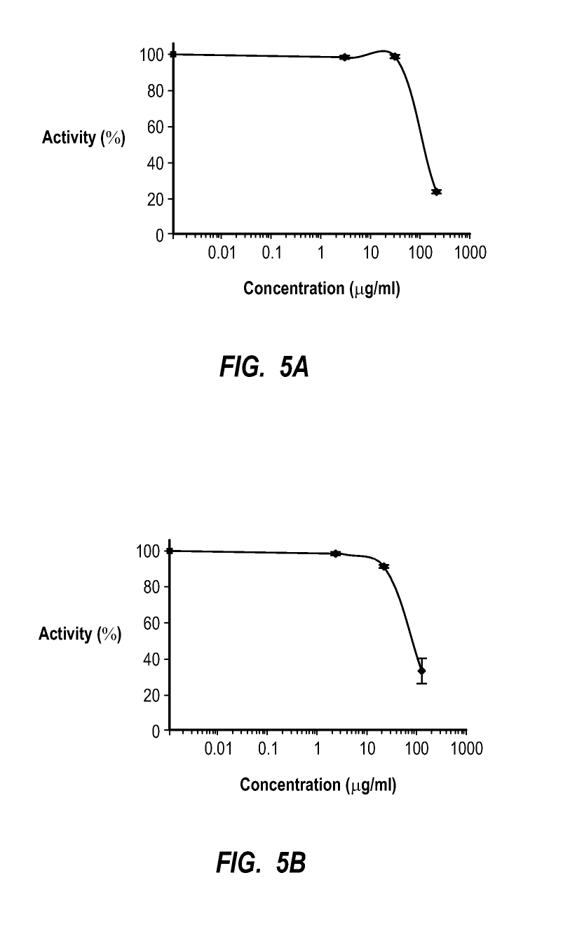

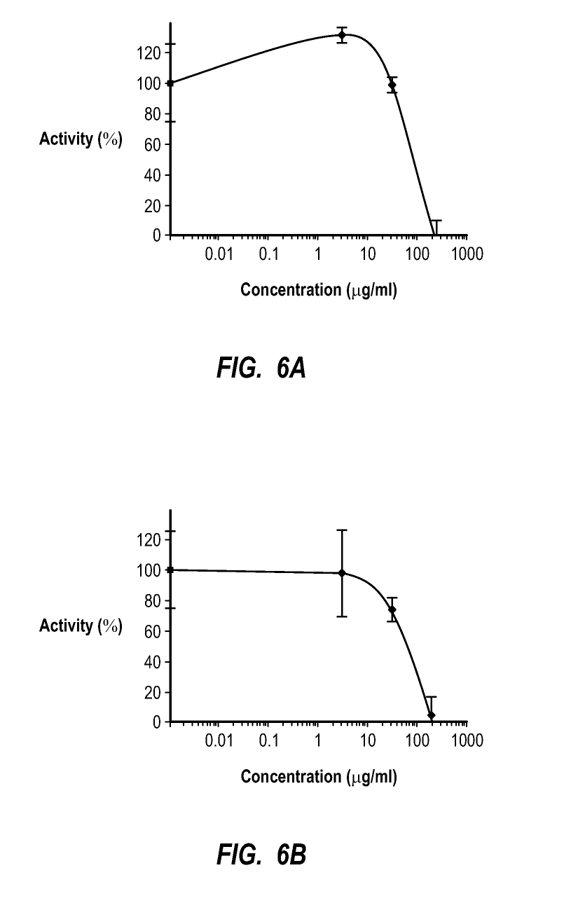

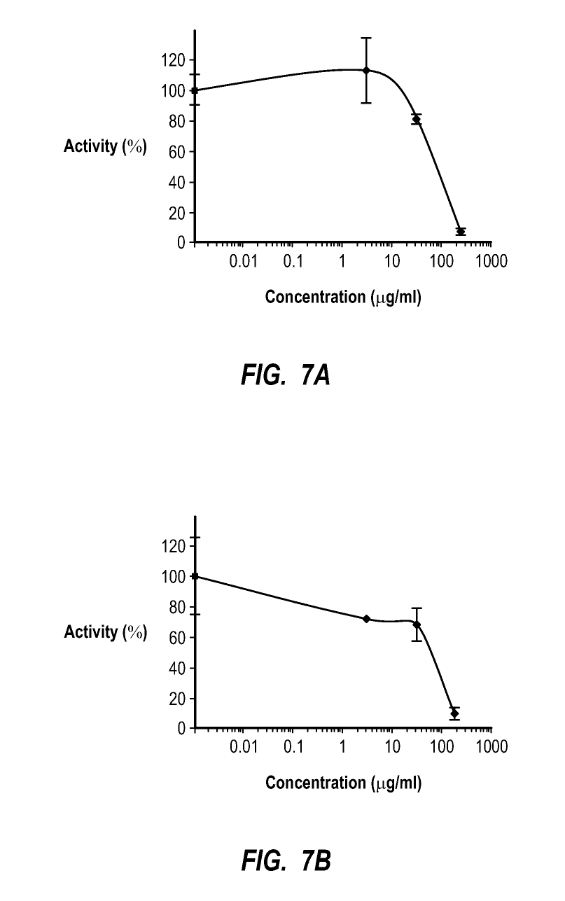

The following tables, charts, and FIGS. 5A, 5B, 6A, 6B, 7A and 7B summarize the assay and the results.

Samples Used

| | | Con- | | |
|---|---|---|---|---|
| | | centration | Extraction | |
| Sample | Form | (mg/ml) | solvent | ViP Number |
| Morinda | Solution | 18.2 | Ethanol | Vip_E_Moci'05_100 |
| citrifolia | | | 80% | |
| Morinda | Solution | 14.5 | Ethanol | Vip_E_Moci'05_100.1 |
| citrifolia | | | 80% | |
| (reproduced) | | | | |

Concentrations

| | Assay | | |
|---|---|---|---|
| | concentrations | Serial | Final solvent |
| Sample number | (μg/ml) | dilution in | concentration |
| Vip_E_Moci'05_100 | 3,30,227 | Ethanol 10% | Ethanol 1% |
| Vip_E_Moci'05_100.1 | 3,30,181 | | |

TNF-α
IC₅₀ Values of TNF-α

| Sample number | IC₅₀ (μg/μl) |
|---|---|
| Vip_E_Moci'05_100 | 100 |
| Vip_E_Moci'05_100.1 | 100 |

Raw Data of TNF-α Inhibition

| Vip_E_Moci'05_100 | | | Vip_E_Moci'05_100.1 | | |
|---|---|---|---|---|---|
| Con- | t (24) | t (0) | Con- | t (24) | t (0) |
| centration | Cytokine | Cytokine | centration | Cytokine | Cytokine |
| (μg/ml) | (pg/ml) | (pg/ml) | (μg/ml) | (pg/ml) | (pg/ml) |
| 227 | 776 | −70^{a)} | 181 | 1021 | 28^{a)} |
| 227 | 818 | * | 181 | 1435 | * |
| 30 | 2958 | * | 30 | 2830 | * |
| 30 | 2921 | * | 30 | 2859 | * |
| 3 | 2926 | 350 | 3 | 3022 | 510 |
| 3 | 2943 | * | 3 | 2958 | * |
| Control | 2873 | * | Control | 2873 | * |
| Control | 2909 | −2^{a)} | Control | 2909 | −2^{a)} |
| Control | 2867 | −12^{a)} | Control | 2867 | −12^{a)} |
| Control | 2859 | * | Control | 2859 | * |
| Control | 2796 | * | Control | 2796 | * |

^{a)}value smaller as the smallest standard curve (62 pg/ml)

IL-1β
IC₅₀ Values of IL-1β

| Sample number | IC₅₀ (μg/μl) |
|---|---|
| Vip_E_Moci'05_100 | 90 |
| Vip_E_Moci'05_100.1 | 60 |

Raw Data of IL-1β Inhibition

| Vip_E_Moci'05_100 | | | Vip_E_Moci'05_100.1 | | |
|---|---|---|---|---|---|
| Con- | t (24) | t (0) | Con- | t (24) | t (0) |
| centration | Cytokine | Cytokine | centration | Cytokine | Cytokine |
| (μg/ml) | (pg/ml) | (pg/ml) | (μg/ml) | (pg/ml) | (pg/ml) |
| 227 | 6 | 24^{a)} | 181 | 33 | 33 |
| 227 | 38 | * | 181 | 62 | * |
| 30 | 138 | 26 | 30 | 136 | 47 |
| 30 | 152 | * | 30 | 118 | * |
| 3 | 176 | * | 3 | 187 | * |
| 3 | 191 | * | 3 | 115 | * |
| Control | 190 | 3^{a)} | Control | 190 | 3^{a)} |
| Control | 148 | 17^{a)} | Control | 148 | 17^{a)} |
| Control | 170 | 18^{a)} | Control | 170 | 18^{a)} |
| Control | 100 | * | Control | 100 | * |
| Control | 123 | * | Control | 123 | * |
| Control | 122 | * | Control | 122 | * |
| Control | 126 | * | Control | 126 | * |

^{a)}value smaller as the smallest standard curve (62 pg/ml)

IC₅₀ Values of IL-6

| Sample number | IC₅₀ (μg/μl) |
|---|---|
| Vip_E_Moci'05_100 | 80 |
| Vip_E_Moci'05_100.1 | 60 |

Raw Data of IL-6 Inhibition

| Vip_E_Moci'05_100 | | | Vip_E_Moci'05_100.1 | | |
|---|---|---|---|---|---|
| Con- | t (24) | t (0) | Con- | t (24) | t (0) |
| centration | Cytokine | Cytokine | centration | Cytokine | Cytokine |
| (μg/ml) | (pg/ml) | (pg/ml) | (μg/ml) | (pg/ml) | (pg/ml) |
| 227 | 105 | 56 | 181 | 149 | 53 |
| 227 | 61 | * | 181 | 74 | * |
| 30 | 855 | 2^{a)} | 30 | 814 | 15 |
| 30 | 798 | * | 30 | 599 | * |
| 3 | 1375 | * | 3 | 754 | * |
| 3 | 953 | * | 3 | 754 | * |
| Control | 1008 | 113 | Control | 1008 | 113 |
| Control | 1129 | 74 | Control | 1129 | 74 |
| Control | 1230 | * | Control | 1230 | * |
| Control | 997 | * | Control | 997 | * |
| Control | 1034 | * | Control | 1034 | * |

^{a)}value smaller as the smallest standard curve (62 pg/ml)

In summary, both extracts induced a concentration dependent inhibition of TNF-α as illustrated above. At an extract concentration of 100 μg/ml a 50% inhibition of TNF-α production was observed. The LPS induced production of cytokine IL-6 was clearly inhibited by ViP_E_{—Moci'}05_{—}100 and ViP_E_Moci'05_{—}100.1 with an IC₅₀ value of 80 μg/ml and 60 μg/ml respectively. A clear inhibition of IL-1βwas also observed with IC₅₀ values of 90 μg/ml ViP_E_Moci'05_{—}100 and 60 μg/ml for ViP_E_Moci'05_{—}100.1.

### Example 4

In another example, another pharmacological screening study was performed to measure the activity spectrum of Morinda citrifolia seed extracts. This study measured the potential inhibitory effect of two extracts on the activity of Human Leukocyte Elastase (HLE). Specifically, the IC₅₀ values were measured on an isolated enzyme for HLE.

Methods utilized to prepare the seed extracts are described in the preceeding examples.

A Leukocyte Elastase Assay was performed as follows. After preincubating the samples with the enzyme HLE (20 nM) at room temperature for 10 minutes, the reaction was started with an enzyme substrate (5 mM). The reaction time was 15 min at room temperature. The controls [t(0)] were performed without enzyme. The effect of several concentrations of sample and reference compounds on the activity of HLE was measured by determining the quantity of p-Nitroaniline under the assay conditions. The quantification of p-Nitroaniline was performed by direct photometrical measurement. The optical densities were measured at λ=415 nm. The quantities were calculated using a standard curve of at least 5 different concentrations.

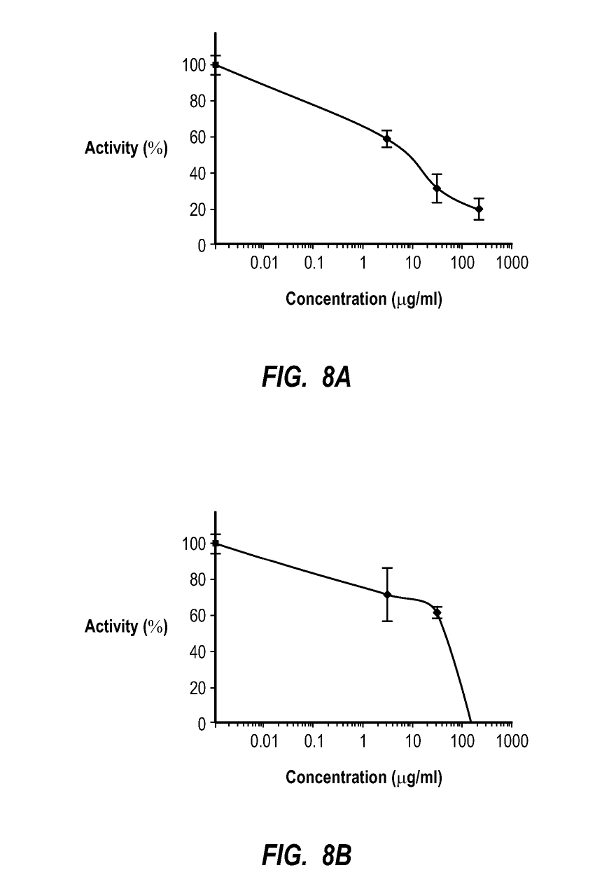

All sample points were measured in duplicate. The dose related inhibition values were expressed as a percentage of the positive control values. The IC₅₀ values (corresponding to the sample concentration at which the inhibition level is 50%) were determined graphically. The following tables, charts, and FIGS. 8A and 8B summarize the assay and the results.

Samples Used

| | | Concen- | | |
|---|---|---|---|---|
| | | tration | Extraction | |
| Sample | Form | (mg/ml) | solvent | ViP Number |
| Morinda | Solution | 18.2 | Ethanol 80% | Vip_E_Moci'05_100 |
| citrifolia | | | | |
| Morinda | Solution | 14.5 | Ethanol 80% | Vip_E_Moci'05_100.1 |
| citrifolia | | | | |
| (reproduced) | | | | |

Concentrations

| | Assay | | |
|---|---|---|---|
| | concentrations | Serial | Final solvent |
| Sample number | (μg/ml) | dilution in | concentration |
| Vip_E_Moci'05_100 | 3,30,227 | Ethanol 10% | Ethanol 1% |
| Vip_E_Moci'05_100.1 | 3,30,181 | | |

HLE
IC₅₀ Values of HLE

| Sample number | IC₅₀ (μg/μl) |
|---|---|
| Vip_E_Moci'05_100 | 7 |
| Vip_E_Moci'05_100.1 | 50 |
| Standard reference agent | IC₅₀ (μM) |
| Ursolic acid | 30 |

Raw Data of HLE Inhibition

| Vip_E_Moci'05_100 | | | Vip_E_Moci'05_100.1 | | |
|---|---|---|---|---|---|
| | t (15) | t (0) | | t (15) | t (0) |
| Concentration | p-NA | p-NA | Concentration | p-NA | p-NA |
| (μg/ml) | (pg/ml) | (pg/ml) | (μg/ml) | (pg/ml) | (pg/ml) |
| 227 | 7.153 | 6.226 | 181 | 4.372 | 4.769 |
| 227 | 6.756 | 6.094 | 181 | 4.637 | 5.100 |
| 30 | 4.703 | 3.379 | 30 | 5.167 | 2.783 |
| 30 | 4.968 | 3.776 | 30 | 5.299 | 2.650 |
| 3 | 5.034 | 2.717 | 3 | 4.902 | 2.584 |
| 3 | 4.902 | 2.452 | 3 | 5.829 | 2.319 |
| Control | 6.557 | 2.386 | Control | 6.557 | 2.386 |
| Control | 6.491 | 2.319 | Control | 6.491 | 2.319 |
| Control | 6.491 | 2.253 | Control | 6.491 | 2.253 |
| Control | 6.292 | 2.518 | Control | 6.292 | 2.518 |

In summary, the two extracts of Morinda citrifolia inhibited the activity of HLE to varying degrees. The ViP_E_Moci'05_{—}100 inhibited the HLE activity with an IC₅₀ value of 7 μg/ml and the ViP_E_Moci'05_{—}100.1 showed a value of 50 μg/ml.

### Example 5

In this example, another pharmacological screening study was performed to measure the activity spectrum of a Morinda citrifolia seed extract. This study measured the potential inhibitory effect of the extract on the activity of inducible Nitrite Oxide Synthase (iNOS). Specifically, the IC₅₀ value was measured on murine macrophages (J774A. 1) for iNOS.

The process for preparing the seed extracts is described in the preceeding examples.

An iNOS Assay was performed as follows. Murine Macrophages (1.5*105 cells/well) were seeded for 24 hours. After preincubating the samples with the cells at room temperature for 10 minutes, the reaction was started with 1 μg/ml LPS (E.coli 055:B5). The incubation time was 24 hours at 37° C. and 5% CO 2. The controls [t(0)] were performed without LPS stimulation.

The quantification of Nitric Oxide was performed with a Nitric Oxide Colorimetric Assay Kit from BioVision (Art.Nr: #K262-200). The optical densities were measured at λ=570 nm. The quantities were calculated using a standard curve of at least 7 different concentrations. Sample points were measured in triplicate.

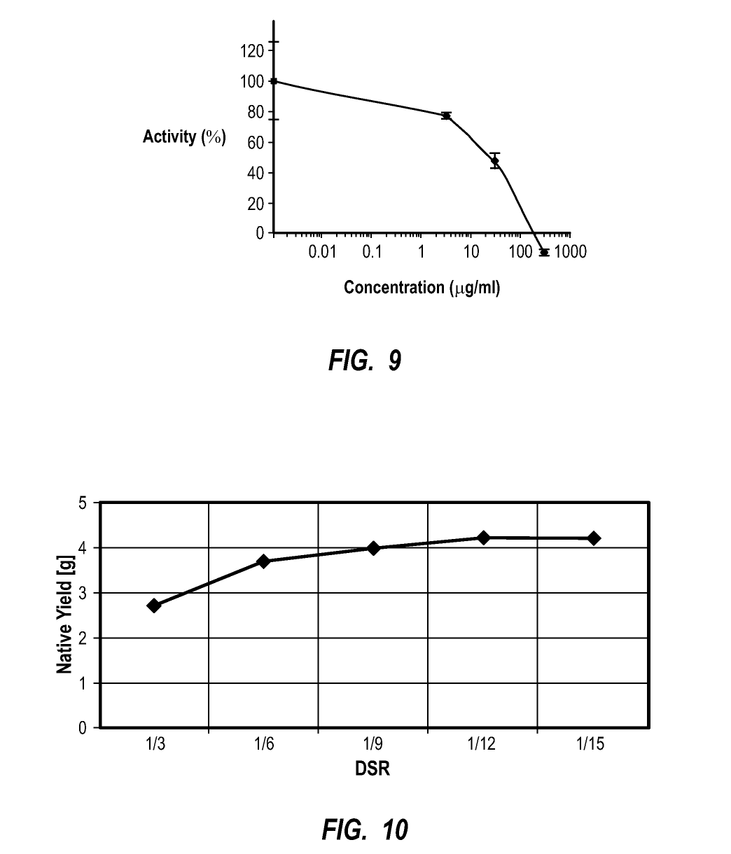

The dose related inhibition values were expressed as a percentage of the positive control values. If applicable, the IC₅₀ values (corresponding to the sample concentration at which the inhibition level is 50%) were determined graphically. The following tables and charts and FIG. 9 summarize the assay and the results.

iNOS
Sample Used

| | | Concen- | | |
|---|---|---|---|---|
| | | tration | Extraction | |
| Sample | Form | (mg/ml) | solvent | ViP Number |
| Morinda | Solution | 18.2 | Ethanol 80% | Vip_E_Moci'05_100 |
| citrifolia | | | | |

IC₅₀ Values of iNOS

| Sample number | IC₅₀ (μg/μl) |
|---|---|
| Vip_E_Moci'05_100 | 25 |
| Standard reference agent | IC₅₀ (μM) |
| L-NAME | 600 |

Raw Data of iNOS Inhibition
Vip_E_Moci'05_100

| Concentration | t(24) | t(0) |
|---|---|---|
| (μg/ml) | iNOS (μM) | iNOS (μM) |
| 300 | 5.0 | 5.9 |
| 300 | 5.2 | |
| 30 | 9.1 | 5.7 |
| 30 | 8.6 | |
| 3 | 10.5 | |
| 3 | 10.9 | |
| 0 | 11.7 | 6.3 |
| 0 | 12.9 | 6.2 |

In summary, the production of Nitric Oxide is catalyzed by the NO-synthases enzyme family (NOS), and distinguishable. The constitutive produced and the inducible isoenzymes of the NO-synthase are distinguishable. The constitutive form —ENOS— occurs in cell types of the cardiovascular system. The inducible type —iNOS— is not produced under basal conditions. The production can be triggered by bacterial lipopolysaccharides or other infective stimuli, during inflammatory diseases, for example in macrophages or endothelial cells. The tested Morinda citrifolia extract Vip_E_Moci'05_{—}100 showed a clear inhibition of iNOS with an IC₅₀ value of 25 μg/ml.

The present invention may be embodied in other specific forms without departing from its spirit of essential characteristics. The described embodiments are to be considered in all respects only as illustrative and not restrictive. The scope of the invention is, therefore, indicated by the appended claims, rather than by the foregoing description. All changes that come within the meaning and range of equivalency of the claims are to be embraced within their scope.

### Example 6

In another example, tests were performed to determine the influence of solvent, 80% w/w and 90% w/w ethanol, on the extraction process. Different process steps were reviewed and adjusted. The amount of necessary auxiliaries was evaluated and defined. The influence of a de-fatting process on the following process steps was tested. Whether the de-fatting process is necessary or not was also investigated. Furthermore a drying process to obtain a free flowing powder was investigated. All process steps were accompanied by pharmacological tests.

All the extractions were made from Morinda Citrifolia seeds milled to 2 mm size in a centrifugal mill. The milled seeds were mixed with 3 parts w/w of n-hexane and stirred for 60 minutes followed by deep layer filtration. The drug residue after filtration was mixed with 2 parts w/w of n-hexane and stirred for 30 minutes. The drug residue was then separated from the hexane by a deep layer filtration. This drug reside obtained was kept over night in an air-flow hood for the removal of residual hexane. This de-fatted drug was the staring material for one set of experiments.

Extracts were made out of the de-fatted and the non de-fatted drug. The solvent used were 80% w/w and 90% w/w ethanol. All extractions were carried out under following fixed parameters in a conical flask with heating and stirring equipment.

| | |
|---|---|
| Temperature: | 40° C. |
| Extraction time: | 120 minutes |
| Drug-solvent-ratio: | 1/6 |
| Amount of Drug: | 100 g |
| Amount of solvent: | 600 g |

After extraction the drug residue was removed by means of deep layer filtration. The filtrates so obtained (miscella) were ready to be concentrated to a soft extract.

In order to concentrate the filtrate, a solution of gum arabicum, 30% w/w in water (with respect to the final dry extract amount) was prepared and then the fluid extract was introduced into this solution during a simultaneous evaporation process under reduced pressure (200-100 mbar) at 40° C. The evaporation process was stopped when a soft extract with a dry content (DC) of about 30% w/w was obtained.

All soft extracts obtained after evaporation were divided in two parts. One part was dried in a lab scale spray-drying unit with an inlet temperature of 170° C. The other part was dried in a vacuum oven under reduced pressure (60-30 mbar) at 40° C.

The de-fatting process was carried and after removal of the residual hexane the drug was used for extraction.

After the extractions and filtration a cloudy filtrate was obtained. There were no noticeable differences between the de-fatted and non de-fatted drugs.

| Extract Number | Drug | Solvent | DC miscella |
|---|---|---|---|
| Vip-E-Moci06-119 | Non de-fatted | 80% w/w EtOH | 1.1% w/w |
| Vip-E-Moci06-120 | Non de-fatted | 80% w/w EtOH | 1.2% w/w |
| Vip-E-Moci06-121 | de-fatted | 80% w/w EtOH | 1.1% w/w |
| Vip-E-Moci06-122 | de-fatted | 90% w/w EtOH | 1.0% w/w |

The dry content (DC) of the miscella was approximately 1% w/w for all 4 extracts.

The simultaneous evaporation process under reduced pressure using a solution of gum arabicum resulted in the formation of a homogeneous soft extract. No precipitations were observed. Extract Vip-E-Moci06-120 showed during the evaporation process the formation of “grease drops”. This problem was solved by increasing the amount of gum arabicum up to 40% w/w (with respect to the final dry extract amount).

| Extract Number | DC (extract incl. gum arabicum) |
|---|---|
| Vip-E-Moci06-119 | 29.6% w/w |
| Vip-E-Moci06-120 | 39.9% w/w |
| | (contains 40% w/w gum arabicum) |
| Vip-E-Moci06-121 | 31.2% w/w |
| Vip-E-Moci06-122 | 27.4% w/w |

The soft extracts were spray dried with an inlet temperature of 170° C. There was no clogging of the nozzle for each of the extracts. No free discharge of powder was obtained. The product completely backed to the inner side of the spray-dryers' tower. Nearly no free powder was reached in the cyclone. Spray drying with the used parameters and in general seems not to be a suitable way for drying.

The vacuum over drying of the soft extracts resulted in a slightly sticky dry cake, which was milled in lab scale mill to get a free flowing powder. For extract number Vip-E-Moci06-120, 1% w/w of silica hydrocolloidalis was added to obtain a free flowing powder. Vacuum oven drying seems to be a feasible way to get a free flowing powder.

In table the pharmacological results for the miscella (fluid extract) and the dry extract of each extraction are represented.

| | | | | Inhibition on 5- |
|---|---|---|---|---|
| | | | | LOX assay IC₅₀ |
| Extract Number | Description | Drug | Solvent | (*) |
| Vip-E-Moci06-119 | Miscella | Non de-fatted | 80% w/w EtOH | 20 μg/ml |
| Vip-E-Moci06-119.3 | Dry extract/spray | Non de-fatted | 80% w/w EtOH | 20 μg/ml |
| Vip-E-Moci06-120 | Miscella | Non de-fatted | 90% w/w EtOH | 25 μg/ml |
| Vip-E-Moci06-120.4 | Dry | Non de-fatted | 90% w/w EtOH | 40 μg/ml |
| | extract/vacuum | | | |
| | oven drying | | | |
| Vip-E-Moci06-121 | Miscella | De-fatted | 80% w/w EtOH | 40 μg/ml |
| Vip-E-Moci06-121.4 | Dry | De-fatted | 80% w/w EtOH | 40 μg/ml |
| | extract/vacuum | | | |
| | oven drying | | | |
| Vip-E-Moci06-122 | Miscella | De-fatted | 90% w/w EtOH | 20 μg/ml |
| Vip-E-Moci06-122.4 | Dry | De-fatted | 90% w/w EtOH | 20 μg/ml |
| | extract/vacuum | | | |
| | oven drying | | | |

Extraction of the non de-fatted drug with 80% w/w ethanol (Vip-E-Moci06-122) resulted in the best pharmacological activities. There was no degradation of activity comparing the miscella (fluid extract) and the final dry extract.

Extract Vip-E-Moci06-119 and Vip-E-Moci06-122 are twice as potent as the other both extracts.

For the extraction and concentration processes, there were no significant differences between the de-fatted and non de-fatted drug. There were also no differences between an 80% w/w and 90% w/w ethanol as extraction solvent. Only the extract with 90% w/w ethanol and the non de-fatted drug needed more auxiliary in the concentration step. Spray drying with used parameters seems to be inapplicable, because no free discharge of product is obtained. The vacuum oven drying method results in a free flowing powder. An extraction with 80% w/w ethanol and the non de-fatted drug (Wip-E-Moci06-119) as well as an extraction with 90% w/w ethanol and the de-fatted drug (Wip-E-Moci06-122) were showing the highest pharmacological activities. According to these results, an extraction of the non de-fatted drug with 80% w/w ethanol could be rated, with regards to economical and technological principles, as best extraction method.

### Example 7

In another example experiments were conducted to determine the optimal processing parameters by factorial experiments. Basic technological parameters were tested in order to ensure that later an economic large scale production could be carried out without technological problems.

In the first experiments, different Drug-Solvent-Rations (DSR) were tested with de-fatted, milled Morindae citrifoliae seeds as starting material in order to determine the best ratio with respect to extractive yield. Futhermore different auxiliaries were tested for their ability to improve the evaporation and drying process.

All actual extractions were made from one Morindae citrifoliae semen sample (Vip-Moci'06-36; provided from Bratt Rawson, 5 kg, Spring, 2006), milled to 2 mm size in centrifugal mill and de-fatted.

One part of the milled seeds were soaked and stirred in 3 parts w/w of hexane for 1 hour followed by deep layer filtration. The drug residue was again soaked and stirred in 2 parts w/w of hexane for 30 minutes followed by deep layer filtration, the defatted drug was kept over night in an air-flow hood for removal of residual hexane. The filtrate was concentrated to determine the actual amount of fatty compounds (hexane soluble). The defatted drug sample was used for the extraction experiments.

All extractions were carried out at 40° C. and for 120 minutes under stirring. The extraction solvent used for all extractions was 80% w/w ethanol.

Drug-Solvent-Ratios (DSR) of 1/3, 1/6, 1/9, 1/12 and 1/15 were tested while maintaining other extraction parameters constant. 50, 0 g of the defatted milled seeds were used for the DSR of 1/3 to 1/15 extractions.

The extractions were carried out in conical flasks with magnetic stirrer and heating. For larger batches (higher DSR) the extraction was carried out in a 2-L reactor with heating and stirring equipment. After extraction the fluid extracts (miscella) were filtered through a deep layer filter and the solvent was evaporated under reduced pressure at 40° C.

Gum arabicum was tested for its attitude to form quasi-emulsions to obtain a homogeneous extract during and after the evaporation process. Therefore the fluid extract was continiously added into a solution of Gum arabicum (10% w/w in water), until the final soft extract was received. The evaporation was carried our under reduced pressure at 40° C.

Gum arabicum was tested for its attitude to form quasi-emulsions to obtain a homogeneous extract during and after the evaporation process. Therefore the fluid extract was continiously added into a solution of Gum arabicum (10% w/w in water), until the final soft extract was received. The evaporation was carried out under reduced pressure at 40° C.

In addition, sodium citrate (tribasic dehydrate; 0,5% w/w of the estimated final extract) was added as a heavy metal chelating agent to one part of the fluid extract containing Gum arabicum in order to prevent a possible loss of biological activity during the evaporation process. The evaporation was carried out under reduced pressure at 40° C.

Silica hydrocolloidalis (Aerosil) was added to the dried extract during the milling process in order to prevent caking and to get a free-flowing powder.

All soft extracts were subsequently dried in a vacuum oven at 40° C. and 100-30 mbar to obtain a dry extract.

The de-fatting process was carried out without any problems. Approx. 6% w/w of fatty compounds (hexane soluble fraction) from the seeds were removed by the de-fatting process.

Extraction and filtration of the fluid extracts revealed no problems. However, during the evaporation of the fluid extracts agglomerations and precipitations appeared which hindered the formation of a homogenous soft extract during evaporation and indicating possible physico-chemical reactions.

The different amounts of yield are shown in the following table.

| | | | Native | |
|---|---|---|---|---|
| Extract Number | DSR | Drug Amount [g] | Yield g | Yield [%] |
| Vip_E_Moci06_102 | 1/3 | 50.0 | 2.7 | 5.5 |
| Vip_E_Moci06_103 | 1/6 | 50.0 | 3.7 | 7.4 |
| Vip_E_Moci06_104 | 1/9 | 50.0 | 4.0 | 8.0 |
| Vip_E_Moci06_105 | 1/12 | 50.0 | 4.2 | 8.4 |
| Vip_E_Moci06_106 | 1/15 | 50.0 | 4.3 | 8.6 |

FIG. 10 shows an increase of yield from DSR 1/3 to 1/12. With a DSR of 1/12 and 1/15 a plateau is reached. There are no relevant differences anymore in the yield between a DSR of 1/12 and 1/15.

Taking the amount of solvent into consideration, a DSR of 1/12 could be rated as suitable under technological and economical aspects for the primary extraction steps. Furthermore the experiments showed, that a standard evaporation procedure will be not suitable to produce a soft extract (which is essential intermediate to produce a dry extract).

The addition of fluid extract to a 10% w/w solution of Gum arabicum (concentration is 20% w/w with respect to final extract) with water, followed by simultaneous evaporation resulted in a homogenous soft extract. No precipitations as seen in the initial experiments were observed anymore.

The addition of sodium citrate (tribasic dehydrate) showed no influence in the evaporation and drying process. The eventual value of sodium citrate will be determined in respect to the biological activity of non treated and treated samples.

The soft extracts were dried in a vacuum oven at 40° C. without a problem. However, the dry extract was sticky and could not be milled to a free flowing powder indicating either hygroscopicity or thermplasticity. This problem could be solved by addition of silica hydrocolloidalis during the milling process. A free flowing powder resulted.

On the basis of the experiments performed so far, the use of the de-fatted rug of 2 mm size, extraction solvent 80% w/w ethanol for 2 hours at 40° C. and a DSR of 1/12 could be set as preliminary parameters for the primary process. For further processing of a soft extract, Gum arabicum seems a suitable additive as it prevented the formation of agglomerates and precipitates during the evaporation process.

In some embodiments, the processing steps maybe manipulated to produce extracts with increased activity. In particular, the concentration of ethanol for the primary extraction may result in a dramatic increase or decrease of activity. For example, extraction with 30% m/m ethanol resulted in IC₅₀ of 100 ug/ml, while extraction with 80% m/m ethanol resulted in a IC₅₀ of 10 ug/ml. Accordingly, in some embodiments, the process steps may be altered to effect the efficacy of inhibition and also maybe altered to allow the production of a bioactive dry powder extract of high potency.

The present invention may be embodied in other specific forms without departing from its spirit of essential characteristics. The described embodiments are to be considered in all respects only as illustrative and not restrictive. The scope of the invention is, therefore, indicated by the appended claims, rather than by the foregoing description. All changes that come within the meaning and range of equivalency of the claims are to be embraced within their scope.

What is claimed is:

1. A method for inhibiting 5-lipoxygenase and 15-lipoxygenase comprising the steps of:
collecting Morinda citrifolia seeds;
pulverizing the seeds;
adding the processed Morinda citrifolia seeds to an alcohol-based solution;
extracting an ingredient from said processed Morinda citrifolia seeds in solution to obtain a fraction;
inhibiting 5-Lipoxygenase and 15-Lipoxygenase by introducing said extracted ingredient to a mammal.

2. The method of claim 1, further comprising the steps of combining the seeds with an organic solvent after being pulverized to defat the seeds and removing excess organic solvent prior to adding the processed seeds to an alcohol-based solution.

3. The method of claim 2, wherein the organic solvent is hexane.

4. The method of claim 2, wherein the seeds are initially defatted in an organic solvent for about one hour at room temperature.

5. The method of claim 2, wherein the steps of defating and removing excess solvent are repeated once prior to extracting a fraction with an alcohol-based solution.

6. The method of claim 1, where in the alcohol based solution is ethanol present in an amount between about 30 and 96% by volume.

7. The method of claim 6, wherein the alcohol based solution is about 80% ethanol.

8. The method of claim 1, further comprising the step of reducing inflammation.

9. The method of claim 1, wherein said alcohol-based solution is comprised of an ingredient selected from the group consisting of methanol, ethanol, and ethyl acetate.

10. The method of claim 1, wherein inhibition of said lipoxygenase is accomplished while maintaining gastric mucosal integrity.

11. A composition for inhibiting 5-Lipoxygenase and 15-Lipoxygenase, said composition comprising a processed Morinda citrifolia component selected from a group consisting of extracts from Morinda citrifolia seeds, Morinda citrifolia seeds, defatted pulverized Morinda citrifolia seed powder.

12. The composition of claim 11 produced in accordance with a method comprising the steps of:
collecting Morinda citrifolia seeds;
pulverizing the seeds;
adding the processed Morinda citrifolia seeds to an alcohol-based solution;
extracting an ingredient from said processed Morinda citrifolia seeds in solution to obtain a fraction.

13. The composition of claim 12, further comprising the steps of combining the seeds with an organic solvent after being pulverized to defat the seeds and removing excess organic solvent prior to adding the processed seeds to an alcohol-based solution.

14. The composition of claim 11, wherein the composition inhibits the synthesis of leukotrienes from arachidonic acid involving the inhibition of one or more Lipoxygenase enzymes.

15. The composition of claim 11, wherein the composition inhibits the oxygenation of arachidonic acid into its intermediate constituents.

16. A method for isolating an active ingredient in a processed Morinda citrifolia product and using said active ingredient to inhibit Lipoxygenase, said method comprising the step of:
obtaining an amount of seeds from a Morinda citrifolia plant;
combining the seeds with an organic solvent;
removing excess organic solvent;
combine defatted seeds with an amount of an alcohol-based solution;
collecting an alcohol soluble fraction;
removing residual alcohol from said alcohol soluble fraction to obtain an alcohol soluble fraction active ingredient;
mixing said active ingredient into a naturaceutical formulation.

17. A method for inhibiting COX-1, COX-2, Interleukin-1β, Interleukin-6, TNF-α, HLE, and iNOS comprising the steps of:
collecting Morinda citrifolia seeds;
pulverizing the seeds;
adding the processed Morinda citrifolia seeds to an alcohol-based solution;
extracting an ingredient from said processed Morinda citrifolia seeds in solution to obtain a fraction;
inhibiting COX-1, COX-2, Interleukin-1β, Interleukin-6, TNF-α, HLE, and iNOS by introducing said extracted ingredient to a mammal.

18. The method of claim 17, further comprising the steps of combining the seeds with an organic solvent after being pulverized to defat the seeds and removing excess organic solvent prior to adding the processed seeds to an alcohol-based solution.

19. The method of claim 18, wherein the organic solvent is hexane.

20. The method of claim 18, wherein the seeds are initially defatted in an organic solvent for about one hour at room temperature.

21. The method of claim 18, wherein the steps of defating and removing excess solvent are repeated once prior to extracting a fraction with an alcohol-based solution.

22. The method of claim 17, where in the alcohol based solution is ethanol present in an amount between about 30 and 96% by volume.

23. The method of claim 22, wherein the alcohol based solution is about 80% ethanol.

24. The method of claim 17, further comprising the step of reducing inflammation.

25. The method of claim 17, wherein said alcohol-based solution is comprised of an ingredient selected from the group consisting of methanol, ethanol, and ethyl acetate.

26. The method of claim 17, wherein inhibition of said COX-1, COX-2, Interleukin-l1β, Interleukin-6, TNF-α, HLE, and iNOS is accomplished while maintaining gastric mucosal integrity.

27. A composition for inhibiting COX-1, COX-2, Interleukin-1β, Interleukin-6, TNF-α, HLE, and iNOS, said composition comprising a processed Morinda citrifolia component selected from a group consisting of extracts from Morinda citrifolia seeds, Morinda citrifolia seeds, defatted pulverized Morinda citrifolia seed powder.

28. The composition of claim 26 produced in accordance with a method comprising the steps of:
collecting Morinda citrifolia seeds;
pulverizing the seeds;
adding the processed Morinda citrifolia seeds to an alcohol-based solution;
extracting an ingredient from said processed Morinda citrifolia seeds in solution to obtain a fraction.

29. The composition of claim 27, further comprising the steps of combining the seeds with an organic solvent after being pulverized to defat the seeds and removing excess organic solvent prior to adding the processed seeds to an alcohol-based solution.

30. The composition of claim 26, wherein the composition inhibits the synthesis of leukotrienes from arachidonic acid involving the inhibition of one or more Lipoxygenase enzymes.

31. The composition of claim 26, wherein the composition inhibits the oxygenation of arachidonic acid into its intermediate constituents.

32. A method for isolating an active ingredient in a processed Morinda citrifolia product and using said active ingredient to inhibit COX-1, COX-2, Interleukin-1β, Interleukin-6, TNF-α, HLE, and iNOS, said method comprising the step of:
obtaining an amount of seeds from a Morinda citrifolia plant;
combining the seeds with an organic solvent;
removing excess organic solvent;
combine defatted seeds with an amount of an alcohol-based solution;
collecting an alcohol soluble fraction;
removing residual alcohol from said alcohol soluble fraction to obtain an alcohol soluble fraction active ingredient;
mixing said active ingredient into a naturaceutical formulation.
